(19) United States
(12) Patent Application Publication
(10) Pub. No.: US 2007/0213242 A1
(43) Pub. Date: Sep. 13, 2007

(54) FOAMING COMPOSITION

(75) Inventor: Odile AUBRUN-SONNEVILLE, Antony (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/671,097

(22) Filed: Feb. 5, 2007

Related U.S. Application Data
(60) Provisional application No. 60/783,844, filed on Mar. 21, 2006.

(30) Foreign Application Priority Data
Mar. 13, 2006 (FR) 06 50834

(51) Int. Cl. A61K 8/00

(52) U.S. Cl. 510/130; 424/70.1

(57) ABSTRACT

A foaming composition suitable for topical application, containing, in an aqueous medium, one or more fatty acid salts in an amount of at least 20% of the total weight of the composition, and at least one surfactant chosen from behenyl alcohol nonionic surfactants and behenic acid nonionic surfactants. The compositions may be in the form of creams or milks, and show very good stability even after heating to very high temperature. They may be used especially in cosmetics or dermatology, as cleansing or makeup-removing products for the skin, the scalp and/or the hair.

### REFERENCE TO PRIOR APPLICATIONS

This application claims priority to U.S. provisional application 60/783,844 filed Mar. 21, 2006, and to French patent application 0650834 filed Mar. 13, 2006, both incorporated herein by reference.

### FIELD OF THE INVENTION

The present invention relates to a soap-based rinsable foaming composition, and also to its use in cosmetics or dermatology, especially as cleansing or makeup-removing products for the skin, the scalp and/or the hair

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing of the skin is very important for facial care, and should be as efficient as possible since greasy residues such as excess sebum, residues of the cosmetic products used daily and makeup products, especially “waterproof” products, accumulate in the folds of the skin and can block the skin pores and lead to the formation of spots.

Several major types of skin cleansing product are known: foaming detergent aqueous lotions and gels, rinsable cleansing anhydrous oils and gels, and foaming compositions, especially foaming creams.

Rinsable anhydrous oils and gels have cleansing action by virtue of the oils contained in these formulations. These oils allow dissolution of the greasy residues and the dispersion of makeup pigments. These products are effective and well tolerated. However, they have the drawback of being heavy, of not foaming and of not giving a sensation of freshness when applied, which is penalizing from a cosmetic viewpoint.

Moreover, foaming detergent aqueous lotions and gels have cleansing action by virtue of surfactants, which place in suspension the greasy residues and the pigments of makeup products. They are effective and pleasant to use due to the fact that they form a foam and can be easily removed. However, lotions are generally quite fluid, which occasionally makes them difficult to handle, and it is difficult to thicken gels while at the same time maintaining good foaming properties.

Foaming compositions containing soaps, which are salts of fatty acids, are known to have good foaming performance and to give good cleansing. These compositions may be in liquid form, as described, for example, in document EP-A-339 994, or in cream form, as described, for example, in documents EP-A-1 166 747 and EP-1 486 558.

The use of creams is preferred by consumers since, unlike liquids, creams do not run when applied, and feel more comfortable on the skin after use. Thus, document EP-A-1 166 747 describes foaming compositions containing soaps, which are in the form of rich, comfortable creams. Although these compositions show good physical stability at room temperature and even up to at least 45° C., microscopic grains may appear in these creams when they are stored at a temperature considerably above 45° C., for example 55° C., and when they are then cooled to room temperature (about 25° C.).

Moreover, the compositions described in EP-1 486 558, which contain a certain proportion of fatty acids, have a granular texture when they are cooled to room temperature after having been heated to 55° C., as shown by the comparative examples presented later.

### SUMMARY OF THE INVENTION

Now, it is advantageous to have available creams that are stable even at high temperature, especially in hot countries or during the hot season in temperate countries. There is thus a need for a foaming composition that remains homogeneous and stable even after storage at a high temperature, especially at temperatures of at least 50° C. and even 55° C.

The inventor has discovered, surprisingly, that the stability of soap-based products is improved by adding a C22 acid- and/or C22 alcohol- (behenic acid or behenyl alcohol) based non-ionic surfactant. The compositions obtained show no phase separation or any formation of grains after storing at high temperature (55° C.) and cooling to room temperature.

The combination of this type of surfactant and soaps provide a composition that is very stable and that gives a foam with good cosmetic qualities and good foaming performance.

### DETAILED DESCRIPTION OF THE PREFERRED EMBODIMENTS

Thus, one subject of the present patent application is a foaming composition suitable for topical application, containing, in an aqueous medium, one or more fatty acid salts in an amount of at least 20% of the total weight of the composition, and at least one surfactant chosen from behenyl alcohol nonionic surfactants and behenic acid nonionic surfactants.

The composition of the invention may be in any form including the form of fluid to thick creams. A cream is a soft product, as opposed to a solid product such as a stick, and it preferably has a viscosity ranging from 3 to 200 poises (0.3 to 20 Pa.s), this viscosity being measured at room temperature (about 25° C.) using a Rheomat RM180 viscometer, 10 minutes after setting the spindle in rotation, at a speed of 200 rpm, the spindle being adapted to the viscosity.

Since the composition according to the invention is preferably intended for topical application, it preferably contains a physiologically acceptable medium. In the present invention, the term “physiologically acceptable medium” means a non-toxic medium that is compatible with human skin (including the interior of the eyelids), mucous membranes, hair or lips. This composition may constitute a cosmetic or dermatological composition, and especially a cosmetic skin-cleansing composition.

The composition according to the invention preferably has a pH that is compatible with topical application, and especially with application to the skin. Its pH preferably ranges from 8 to 9.8 and better still from 8.5 to 9.6.

### Fatty Acid Salt

Fatty acid salts, or soaps, useful herein may be obtained from a fatty acid and a base, the fatty acid comprising for example a linear or branched, saturated or unsaturated alkyl chain containing from 12 to 22 carbon atoms and preferably from 12 to 20 carbon atoms.

The bases (also known as saponifying agents) totally or partially neutralize the fatty acids. The bases that may be used to obtain the salts inlcude, for example, mineral bases, for instance alkali metal hydroxides (sodium hydroxide and potassium hydroxide), alkaline-earth metal hydroxides (magnesium hydroxide) or ammonium hydroxide, or alternatively organic bases, for instance triethanolamine, N-methylglucamine, lysine and arginine. According to one particular embodiment of the invention, the base is potassium hydroxide.

The soap is generally introduced into the composition in the form of the base, on the one hand, and/or of the fatty acid, on the other hand, the formation of the salt taking place in situ.

The degree of neutralization of the fatty acid is defined as being the mass ratio between the fatty acids in the form of salts and the total fatty acids (free acids+fatty acid salts). In the composition according to the invention, this degree preferably ranges from 80% to 97% and better still from 85% to 95%.

The fatty acid may be chosen in particular from C10 to C24 and especially C12-C18 fatty acids, and in particular lauric acid, myristic acid, stearic acid and palmitic acid, and mixtures thereof.

Examples of soaps that may be mentioned include the potassium salts and sodium salts of C10 to C24, especially C12-C20 and more especially C12-C18 fatty acids. The soap may be chosen more especially from the potassium salts of C12-C18 fatty acids, more especially the potassium salt of lauric acid, the potassium salt of myristic acid, the potassium salt of palmitic acid or the potassium salt of stearic acid, and mixtures thereof.

The total amount of fatty acid salts in the composition of the invention is preferably at least 20% of the total weight of the composition. It may range, for example, from 20% to 40% by weight, preferably from 25% to 40% by weight and better still from 25% to 35% by weight relative to the total weight of the composition.

According to one preferred embodiment of the invention, the composition contains an amount of salts of C12-C14 fatty acids, especially of lauric acid and of myristic acid, of at least 10% by weight and better still of at least 15% by weight relative to the total weight of the composition.

### Behenyl Alcohol Nonionic Surfactants and Behenic Acid Nonionic Surfactants

Behenic acid and behenyl alcohol contain 22 carbon atoms. The invention C22 acid and C22 alcohol nonionic surfactants are derivatives of behenic acid and behenyl alcohol and do not comprise any of the C22 soaps described above (i.e., are not any of the fatty acid salts described above) and may especially be a compound comprising a polar portion chosen from oxyethylenated units, oxyethylenated and oxypropylenated units, polyglycerolated units, sugar units such as glucose, and mixtures thereof. According to one preferred embodiment of the invention, the C22 nonionic surfactant is chosen from oxyethylenated derivatives of behenic acid or of behenyl alcohol, especially those containing from 5 to 100 oxyethylene groups.

Preferably, the C22 nonionic surfactant is chosen from oxyethylenated behenyl alcohol derivatives containing from 5 to 100 oxyethylene groups and preferably from 5 to 50 oxyethylene groups, such as Beheneth-10, for instance the product sold under the name Eumulgin BA 10 by the company Cognis or under the name Emalex BHA-10 by the company Nihon Emulsion, or Beheneth-20, for instance the product sold under the name Nikkol BB 20 by the company Nikko Chemicals. According to one preferred mode of the invention, the nonionic surfactant chosen from behenyl alcohol and behenic acid derivatives is chosen from Beheneth-10 (behenyl alcohol derivative with 10 oxyethylene units) and Beheneth-20 (behenyl alcohol derivative with 20 oxyethylene units), and mixtures thereof.

The amount of behenyl alcohol nonionic surfactant(s) and/or behenic acid nonionic surfactant(s) may range, for example, from 0.5% to 10% by weight and preferably from 1% to 5% by weight relative to the total weight of the composition, including 2, 3, 4, 6, 7, 8, and 9% and all subranges and values between stated values.

### Other Surfactants

The composition according to the invention may also contain one or more additional surfactants, i.e. other than the fatty acid salts and the behenic acid and/or behenyl alcohol non-ionic surfactants. These additional surfactants may be chosen for example from nonionic surfactants, amphoteric or zwitterionic surfactants, anionic surfactants and cationic surfactants, and mixtures thereof. When they are present, their concentration may range from 0.1% to 20% of the total weight of the composition and preferably from 0.1% to 10% of the total weight of the composition.

Examples of nonionic surfactants that may be used include alkyl polyglucosides (APG), maltose esters, polyglycerolated fatty alcohols, and glucamine derivatives, for instance 2-ethylhexyloxycarbonyl-N-methylglucamine, and mixtures thereof.

Alkylpolyglucosides that are preferably used include those containing an alkyl group containing from 6 to 30 carbon atoms and preferably from 8 to 16 carbon atoms, and containing a hydrophilic group (glucoside) preferably comprising 1.2 to 3 saccharide units. Examples of alkylpolyglucosides that may be mentioned include decylglucoside (Alkyl-C9/C11-polyglucoside (1.4)), for instance the product sold under the name Mydol 10® by the company Kao Chemicals, the product sold under the name Plantaren 2000 UP® by the company Cognis, and the product sold under the name Oramix NS 10® by the company SEPPIC; caprylyl/capryl glucoside, for instance the product sold under the name Oramix CG 110® by the company SEPPIC; lauryl glucoside, for instance the products sold under the names Plantaren 1200 N® and Plantacare 1200® by the company Cognis; cocoglucoside, for instance the product sold under the name Plantacare 818/UP® by the company Cognis.

The maltose derivatives are, for example, those described in document EP-A-566 438, such as O-octanoyl-6′-D-maltose or O-dodecanoyl-6′-D-maltose described in document FR-A-2 739 556.

Among the polyglycerolated fatty alcohols that may be mentioned is polyglycerolated (3.5 mol of glycerol) dodecanediol, this product being sold under the name Chimexane NF® by the company Chimex.

The amphoteric and zwitterionic foaming surfactants may be chosen, for example, from betaine derivatives including amidopropylbetaines, amphoacetates and hydroxylsultaines, and mixtures thereof.

Examples of betaine derivatives that may be mentioned include, for example, cocobetaine, for instance the product sold under the name Dehyton AB-30® by the company Cognis; laurylbetaine, for instance the product sold under the name Genagen KB® by the company Clariant; oxyethylenated (10 EO) laurylbetaine, for instance the product sold under the name Lauryl ether (10 OE) betaine® by the company Shin Nihon Rica; oxyethylenated (10 EO) stearylbetaine, for instance the product sold under the name Stearyl ether (10 OE) betaine® by the company Shin Nihon Rica, the cocamidopropyl betaine sold, for example, under the name Velvetex BK 35® by the company Cognis, or the undecylenamidopropyl betaine sold, for example, under the name Amphoram U® by the company Ceca.

Examples of alkyl amphoacetates that may be mentioned include N-disodium N-cocoyl-N-carboxymethoxyethyl-N-carboxymethylethylenediamine (INCI name: disodium cocamphodiacetate), for instance the product sold under the name Miranol C2M Concentrate NP® by the company Rhodia Chimie, and N-sodium N-cocoyl-N-hydroxyethyl-N-carboxymethylethylenediamine (INCI name: sodium cocamphoacetate).

The anionic surfactants may be chosen, for example, from sarcosinates and acyl sarcosinates and salts thereof, such as sodium lauroyl sarcosinate; taurates and methyltaurates and salts thereof; isethionates and acyl isethionates, products of reaction of fatty acids containing from 10 to 22 carbon atoms with isethionic acid, and salts thereof such as sodium isethionate and sodium cocoyl isethionate; sulfosuccinates and salts thereof; alkyl sulfates and alkyl ether sulfates and salts thereof, especially sodium or triethanolamine lauryl sulfate, and sodium or potassium lauryl ether sulfate; monoalkyl and dialkyl esters of phosphoric acid and salts thereof, for instance sodium mono- and dilauryl phosphate, potassium mono- and dilauryl phosphate, triethanolamine mono- and dilauryl phosphate, sodium mono- and dimyristyl phosphate, potassium mono- and dimyristyl phosphate, diethanolamine mono- and dimyristyl phosphate, and triethanolamine mono- and dimyristyl phosphate; alkane sulfonates and salts thereof; bile salts such as cholates, deoxycholates, taurocholates and taurodeoxycholates; lipoamino acids and salts thereof, such as monosodium and disodium acylglutamates; and mixtures thereof.

According to one particular embodiment of the invention, the composition contains at least one nonionic surfactant chosen from alkylpolyglucosides, as additional surfactant.

### Additives

The aqueous medium of the foaming composition of the invention, which is preferably intended for topical application, is preferably a physiologically acceptable medium, and it generally contains water, which may represent at least 10% of the total weight of the composition and preferably at least 20% of the total weight of the composition. The amount of water may range, for example, from 10% to 75% by weight, preferably from 20% to 70% by weight and better still from 20% to 60% by weight relative to the total weight of the composition.

The composition may contain, besides water, one or more solvents chosen from lower alcohols containing from 1 to 6 carbon atoms, such as ethanol; polyols such as glycerol; glycols, for instance butylene glycol, isoprene glycol, propylene glycol or polyethylene glycols such as PEG-8; sorbitol; sugars such as glucose, fructose, maltose, lactose and sucrose; and mixtures thereof. The amount of solvent(s) in the composition of the invention may range from 0.5% to 50% by weight and preferably from 5% to 40% by weight relative to the total weight of the composition.

According to one preferred embodiment of the invention, the composition contains one or more polyols, and preferably at least 5% by weight of one or more polyols, relative to the total weight of the composition.

The composition of the invention may also comprise any additive or active agent such as those conventionally used in products for topical application, especially cosmetic or dermatological products. Examples that may be mentioned include water-soluble or liposoluble adjuvants that are common in cosmetics or dermatology, such as preserving agents, sequestrants (EDTA), antioxidants, fragrances, dyestuffs, soluble dyes or encapsulated or non-encapsulated pigments, nacres, fillers (fillers with matting, tensioning, whitening or exfoliant effects), sunscreens, polymers, and hydrophilic or lipophilic cosmetic or dermatological active agents.

The amounts of these various adjuvants are those conventionally used in the field under consideration, for example from 0.01% to 20% of the total weight of the composition. These adjuvants and the concentrations thereof should preferably be such that they do not overly negatively modify the property desired for the composition of the invention.

As fillers that may be used in the composition of the invention, examples that may be mentioned include silica and in particular silica microbeads such as those sold under the name SB150 by the company Miyoshi; talc; kaolin; the glass and ceramic beads sold by the company 3M under the trade name Macrolite; polyamide particles and especially those sold under the name Orgasol by the company Atochem; non-expanded synthetic polymer powders, such as polyethylene, polystyrene, polyester or polyamide (for example Nylon or poly-β-alanine) powders, powders of acrylate copolymers (for example the microporous microspheres sold by the company Dow Corning under the trade name Polytrap), polymethacrylic acid powders, Teflon® (polytetrafluoro-ethylene or PTFE) powders, for instance the products sold under the name Fluon by the company Uniqema and the product sold under the name Fluoropure 812C by the company Shamrock (PTFE powder with a particle size of 12 μm); expanded powders such as hollow thermoplastic microspheres prepared via known processes, for instance those described in documents U.S. Pat. No. 3,615,972 and EP-A-0 56219, and especially the microspheres sold under the name Expancel by the company Kemanord Plast or under the name Micropearl F 80 ED by the company Matsumoto; powders of natural organic materials such as crosslinked or non-crosslinked, modified or unmodified starch (corn starch, wheat starch or rice starch) powders, such as the starch powders crosslinked with octenylsuccinate anhydride sold under the name Dry-Flo by the company National Starch; silicone resin microbeads such as those sold under the name Tospearl by the company Toshiba Silicone; and mixtures thereof.

Examples of active agents that may be mentioned include water-soluble or liposoluble vitamins, such as vitamin A (retinol), vitamin C (ascorbic acid), vitamin E (tocopherol), vitamin B3 (or vitamin PP or niacinamide), vitamin B5 (panthenol in its various forms: D-panthenol, DL-panthenol), vitamin D, vitamin F (mixture of essential fatty acids), derivatives, precursors and analogues of these vitamins; α-hydroxy acids, especially fruit-based acids, for instance glycolic acid, lactic acid, malic acid, citric acid, tartaric acid and mandelic acid, and derivatives and mixtures thereof; β-hydroxy acids, for instance salicylic acid and derivatives thereof such as 5-n-octanoylsalicylic acid or 5-n-dodecanoylsalicylic acid; antiseptics; anti-seborrhoeic and antimicrobial agents such as benzoyl peroxide, salicylic acid, 2,4,4′-trichloro-2′-hydroxydiphenyl ether (or triclosan), 3,4,4′-trichlorocarbanilide (or triclocarban), azelaic acid and zinc salts such as zinc gluconate; slimming agents such as caffeine; optical brighteners; electrolytes; and in general any agent that has the effect of improving the cosmetic properties of the skin.

The polymers that may be used in the composition of the invention may be hydrophilic or lipophilic, anionic, nonionic, cationic or amphoteric, thickening or dispersing.

Examples of polymers that may be mentioned include polysaccharides, for instance xanthan gum, guar gum, alginates and modified celluloses; synthetic polymers such as acrylic polymers and copolymers, acrylamide-based polymers and copolymers, polyalkylene glycols, preferably polyethylene glycols, of high molecular weight, and mixtures of these polymers.

The high molecular weight polyethylene glycols have a number of ethylene oxide (EO) units of greater than 10, preferably of at least 50 and better still of at least 100, for instance polyethylene glycol containing 14 000 EO (CTFA name: PEG-14 M), polyethylene glycol containing 7000 EO (CTFA name: PEG-7 M), polyethylene glycol containing 20 000 EO (CTFA name: PEG-20 M) and polyethylene glycol containing 60 000 EO (CTFA name: PEG-60 M).

Polymers that may also be mentioned include cationic polymers of the Polyquaternium type, which give the foaming composition softness and creaminess. These cationic polymers may preferably be chosen from the following polymers:
Polyquaternium 5, such as the product Merquat 5 sold by the company Calgon;
Polyquaternium 6, such as the product Salcare SC 30 sold by the company Ciba, and the product Merquat 100 sold by the company Calgon;
Polyquaternium 7, such as the products Merquat S. Merquat 2200 and Merquat 550 sold by the company Calgon; and the product Salcare SC 10 sold by the company Ciba;
Polyquaternium 10, such as the product Polymer JR400 sold by the company Amerchol;
Polyquaternium 11, such as the products Gafquat 755, Gafquat 755N and Gafquat 734 sold by the company ISP;
Polyquaternium 15, such as the product Rohagit KF 720 F sold by the company Rohm;
Polyquaternium 16, such as the products Luviquat FC905, Luviquat FC370, Luviquat HM552 and Luviquat FC550 sold by the company BASF;
Polyquaternium 22, such as the product Merquat 280 sold by the company Calgon;
Polyquaternium 28, such as the product Styleze CC10 sold by the company ISP;
Polyquaternium 39, such as the product Merquat Plus 3330 sold by the company Calgon;
Polyquaternium 44, such as the product Luviquat Care sold by the company BASF;
Polyquaternium 46, such as the product Luviquat Hold sold by the company BASF;
Polyquaternium 47, such as the product Merquat 2001 sold by the company Calgon.

Cationic polymers that may also be used include cationic guars, such as the product Jaguar sold by the company Rhodia.

The compositions according to the invention may especially constitute cleansing or makeup-removing products for the skin (body or face, including the eyes), the scalp and/or the hair. They may more particularly constitute a skin cleansing composition.

These compositions develop a foam when they are mixed with water. They may be used several ways, including:
spreading the composition between the hands, applying it to the face or the body, etc., and then massaging it in the presence of water to develop the foam directly on the face or the body,
developing the foam in the palms of the hands before applying the foam obtained to the face or the body, etc.

In both cases, the foam is preferably then rinsed off.

Another subject of the invention is the cosmetic use of the composition as defined above, as a cleansing and/or makeup-removing product for the skin, the scalp and/or the hair.

Another subject of the invention is a process for cleansing the skin, the scalp and/or the hair, wherein the composition of the invention is applied to the skin, the scalp and/or the hair, in the presence of water, and the foam formed and the soiling residues are removed by rinsing with water.

The examples that follow serve to illustrate the invention without, however, being limiting in nature. The amounts indicated are in weight percentages, unless otherwise mentioned.

| | | Com- | Com- | Com- |
|---|---|---|---|---|
| | | parative | parative | parative |
| | Example 1 | Example 1 | Example 2 | Example 3 |
| Lauric acid | 3 | 3 | 3 | 3 |
| Myristic acid | 20 | 20 | 20 | 20 |
| Stearic acid | 6 | 6 | 6 | 6 |
| Glyceryl stearate | 2 | 2 | 2 | 2 |
| Decyl glucoside | 1 | 1 | 1 | 1 |
| Potassium | 6.2 | 6.2 | 6.2 | 6.2 |
| hydroxide | | | | |
| Glycerol | 14 | 14 | 14 | 14 |
| PEG-8 | 7 | 7 | 7 | 7 |
| Beheneth-10 | 1 | — | — | — |
| Behenic acid | — | — | 1 | — |
| Behenyl alcohol | — | — | — | 1 |
| Preserving agents | 0.9 | 0.9 | 0.9 | 0.9 |
| Water | qs 100 | qs 100 | qs 100 | qs 100 |
| pH | 9.3 | 9.3 | 9.4 | 9.3 |
| Appearance after | Homogeneous | Granular | Granular | Granular C |
| storage for 2 | texture | | | |
| months under hot | | | | |
| conditions | | | | |
| (at 55° C.) and | | | | |
| cooling to room | | | | |
| temperature | | | | |

In these examples, the degree of neutralization of the fatty acids is about 90%.

### Procedure:

The fatty acids, the Beheneth-10 and the glyceryl stearate were dispersed in the aqueous phase comprising the water, the polyols and the preserving agents, using a rotor-stator turbomixer. They were then neutralized with potassium hydroxide at 80° C. The mixture was slowly cooled with paddle stirring, and the decyl glucoside was added at about 40° C. The mixture was then paddle-blended until cooling to 25° C. was complete.

The comparative examples 1 to 3 have a granular appearance after storage at a temperature of 55° C. These comparative examples show that replacing the behenyl alcohol-based surfactant with behenic acid or behenyl alcohol does not allow the aim of the invention to be achieved.

The above written description of the invention provides a manner and process of making and using it such that any person skilled in this art is enabled to make and use the same, this enablement being provided in particular for the subject matter of the appended claims, which make up a part of the original description and including a foaming composition, optionally for topical application, containing, in an aqueous medium, one or more fatty acid salts in an amount of at least 20% of the total weight of the composition, and at least one nonionic surfactant chosen from behenyl alcohol derivatives and behenic acid derivatives, and mixtures thereof, meaning at least one C22 acid and/or C22 alcohol nonionic surfactant as described above.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A foaming composition suitable for topical application, comprising, in an aqueous medium, one or more fatty acid salts in an amount of at least 20% of the total weight of the composition, and at least one surfactant chosen from behenyl alcohol nonionic surfactants and behenic acid nonionic surfactants.

2. The composition according to claim 1, wherein the pH ranges from 8 to 9.8.

3. The composition according to claim 1, wherein the degree of neutralization of the fatty acid ranges from 80% to 97%.

4. The composition according to claim 1, wherein the fatty acid is chosen from lauric acid, myristic acid, stearic acid and palmitic acid, and mixtures thereof.

5. The composition according to claim 1, wherein the fatty acid salt is chosen from the potassium salts of C12-C18 fatty acids.

6. The composition according to claim 1, wherein the total amount of fatty acid salts ranges from 20% to 40% by weight relative to the total weight of the composition.

7. The composition according to claim 1, comprising an amount of C12-C14 fatty acid salts of at least 10% by weight relative to the total weight of the composition.

8. The composition according to claim 1, comprising at least one behenyl alcohol nonionic surfactant and/or at least one behenic acid nonionic surfactant comprising a polar portion chosen from oxyethylenated units, oxyethylenated and oxypropylenated units, polyglycerolated units and sugar units, and mixtures thereof.

9. The composition according to claim 1, comprising at least one behenyl alcohol nonionic surfactant and/or at least one behenic acid nonionic surfactant that is an oxyethylenated derivative containing from 5 to 100 oxyethylene groups.

10. The composition according to claim 1, comprising at least one behenyl alcohol nonionic surfactant comprising from 5 to 100 oxyethylene groups.

11. The composition according to claim 1, wherein the amount of the at least one surfactant chosen from behenyl alcohol nonionic surfactants and behenic acid nonionic surfactants is 0.5% to 100 by weight relative to the total weight of the composition.

12. The composition according to claim 1, further comprising one or more additional surfactants chosen from nonionic surfactants, amphoteric or zwitterionic surfactants, anionic surfactants and cationic surfactants, and mixtures thereof.

13. The composition according to claim 12, further comprising one or more alkyl polyglucosides.

14. The composition according to claim 12, wherein the amount of additional surfactants is 0.1% to 20% by weight relative to the total weight of the composition.

15. The composition according to claim 1, wherein said composition is a skin cleansing composition.

16. A process, comprising applying the composition according to claim 1 to at least one of the skin, the scalp and the hair.
